(19) United States
(12) Patent Application Publication
(10) Pub. No.: US 2014/0357619 A1
(43) Pub. Date: Dec. 4, 2014

(54) Apparatus for applying health treatment.

(71) Applicant: Edric Sizemore, Richton Park, IL (US)

(72) Inventor: Edric Sizemore, Richton Park, IL (US)

(21) Appl. No.: 14/282,027

(22) Filed: May 20, 2014

Related U.S. Application Data
(60) Provisional application No. 61/830,123, filed on Jun. 2, 2013.

(51) Int. Cl. A61K 31/4402; A61Q 19/00; A61K 8/49

(52) U.S. Cl. CPC A61K 31/4402; A61K 8/4926; A61Q 19/001; A61K 2800/74
USPC 514/212.01; 401/265

(57) ABSTRACT

An apparatus to apply antiseptic treatment while providing the benefits of a moisturizer. A method for using the apparatus whereby the antiseptic treatment is applied to a body orifice via a squeeze tube, a twist up stick tube or by using a finger to retrieve the antiseptic treatment from a small jar with a twist-off top to help provide a protectant that reduces a body's intake of harmful microbes.

### CROSS-REFERENCE TO RELATED APPLICATIONS

The present application claims the benefit of U.S. Provisional Patent Application No. 61/830,123, filed Jun. 2, 2013, the entirety of which is hereby incorporated by reference.

### BACKGROUND

Every year, millions of people around the world are exposed to several contagious germs and bacteria that cause flu and cold symptoms. News reports on the increasing number of people contracting the flu or cold during the peak season continue to consume television and radio. One problem that exists with exposure to infected people is the inability for an unaffected person to quickly and conveniently protect and shield themselves against contracting the flu, cold or rhinovirus germs.

The present invention relates to a moisturizing lip balm antiseptic treatment that fights and kills infectious cold or flu germs and bacteria on contact with the lips. The present invention also teaches a method for reducing and preventing the chances of a person contracting the cold and flu virus when exposed to infectious people by providing a convenient and simple method of applying the moisturizing lip balm antiseptic to a body part, such as a person's lips to name one example, via a squeeze tube, a twist up lip stick tube or using a finger to retrieve from a small jar with a twist-off top.

Body orifices, such as the mouth, are one of the most common ways a person can take in cold or flu germs into the body. Millions of people are infected each year by, for example, coming in contact with cold or flu germs on the lips from an infected hand touching the mouth, the lips coming in contact with an infected glass, cup, bottle or airborne germs landing on the lips from an infected person coughing or sneezing.

The moisturizing lip balm antiseptic treatment of the present invention contains an active ingredient, such as cetylpyridinium chloride, scientifically proven to fight and kill cold, flu and rhinovirus germs and bacteria on contact. The active ingredient is combined with moisturizing lip balm ingredients such as, but not limited to, Petrolatum, Lanolin, Cetyl Alcohol, Coco Butter, Microcrystillen Wax SP-19, Bees Wax and Menthol.

The present invention is convenient to apply. It being applied by using an easy to carry dispenser such as a squeeze tube, a twist up lip stick tube, or a small jar with a twist-off top, to name a few examples.

The present invention provides dual benefits with each use. It's a moisturizing lip balm to protect chapped or dry lips, and an active anti-infective antiseptic treatment that fights and kills infectious cold and flu germs and bacteria on contact with the body. It is well known in the art that infectious germs can be neutralized or killed once inside the body, in the mouth, at the back of the throat for instance. However, these infectious germs can still lie dormant on the body exterior. This creates a problem if infectious germs are living on the lips nostril area for instance. A person can touch their infected areas and subsequently touch their eyes, thus allowing the virus to enter the blood stream through the eyes and infecting the person with the germs. The present invention addresses this problem.

For the foregoing reasons, there is a need in the industry for an apparatus and method for using that apparatus that provides a moisturizing balm, and also acts as an active anti-infective antiseptic treatment that fights and kills infectious cold and flu germs and bacteria on contact with body orifices.

### SUMMARY

In accordance with the forgoing drawbacks associated with the prior art, it is an object of the present invention to provide a moisturizing lip balm antiseptic treatment that fights and kills infectious cold or flu germs and bacteria on contact with the lips.

It is an object of the present invention to provide dual benefits in one product: it's a moisturizing lip balm to protect chapped or dry lips, and an active anti-infective antiseptic treatment that fights and kills infectious cold and flu germs and bacteria on contact with the lips.

It is a further object of the present invention to provide a moisturizing lip balm antiseptic treatment that contains an active ingredient, cetylpyridinium chloride, scientifically proven to fight and kill cold, flu and rhinovirus germs and bacteria on contact. The active ingredient, such as Cetylpyridinium chloride or the like, is combined with moisturizing lip balm ingredients such as, but not limited to, Petrolatum, Lanolin, Cetyl Alcohol, Coco Butter, Microcrystillen Wax SP-19, Bees Wax and Menthol.

It is another object of the present invention to provide a moisturizing lip balm antiseptic treatment that is quickly applied by using an easy to carry dispenser such as, squeeze tube, a twist up lip stick tube or a small 0.25 oz jar with a twist-off top. These dispensers are not limited to 0.25 ounces of combined ingredients and are far more convenient and accessible to use than the prior art.

Yet another object of the present invention is to provide a moisturizing lip balm antiseptic treatment that may be applied to the outer area of the nostrils and inner edges to coat the areas and the inner nostril hairs with the antiseptic to help provide a protectant that reduces the inhalation of live infectious airborne cold and flu germs.

Another object of the present invention is to provide a moisturizing lip balm to heal and protect chapped or dry lips from the wind, sun and other elements that cause chapped or dry lips.

It is still a further object of the present invention to provide a quick and convenient method to reduce and prevent the chances of a person contracting the cold and flu virus when exposed to infectious people.

These and additional objects of the present invention may be determined from a review of the instance disclosure, disclosing a moisturizing lip balm antiseptic treatment that heals and protects chapped or dry lips, but more importantly fights and kills infectious cold or flu germs and bacteria on contact with the lips, thus reducing and preventing the chances of a person contracting the cold and flu germs at the mouth when exposed to infectious people.

The above brief description sets forth the more important features and benefits of the present disclosure so that the description that follows may be better understood. It is to be understood that the disclosure is not limited in its application to the details of the construction and the arrangements set forth in the following description or illustration in the drawings. Also, it is understood that the phraseology and terminology employed herein are for description and not limitation.

### BRIEF DESCRIPTION OF THE SEVERAL VIEWS OF THE DRAWINGS

These and other features, aspects and advantages of the present invention, which has actually been reduced to practice by the inventor in one embodiment, will become better understood with regard to the following description, appended claims, and accompanying drawings where:

FIG. 1 shows a perspective view of an apparatus embodying features of an of the invention.

FIG. 2 shows a perspective view of the apparatus embodying features of an example of the invention and showing the apparatus with treatment being applied by a person.

FIG. 3 shows a perspective view of the apparatus embodying features of another example of the invention and showing the apparatus with treatment being applied by a person.

### DESCRIPTION

### Overview

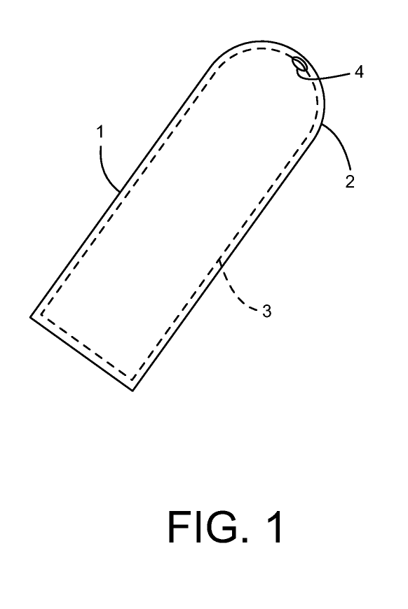

As shown in FIG. 1, an apparatus embodying features of an example of the invention comprises a container 1, a treatment composition 3, the treatment composition 3 being located within the container 1, the container 1 having an expelling part 2, the expelling part 2 having an opening 4.

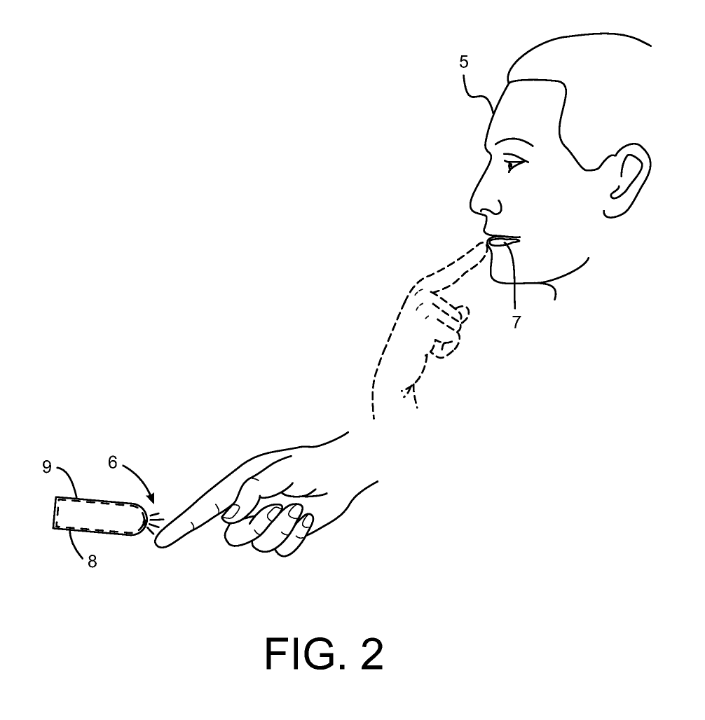

As shown in FIG. 2, an apparatus embodying features of an example of the invention and showing the apparatus with treatment being applied by a person comprises a container 9, treatment composition 8 within the container 9. A person 5 taking the treatment composition 8 expelled from 6 the container 9 and applying it as balm upon the person's lips 7.

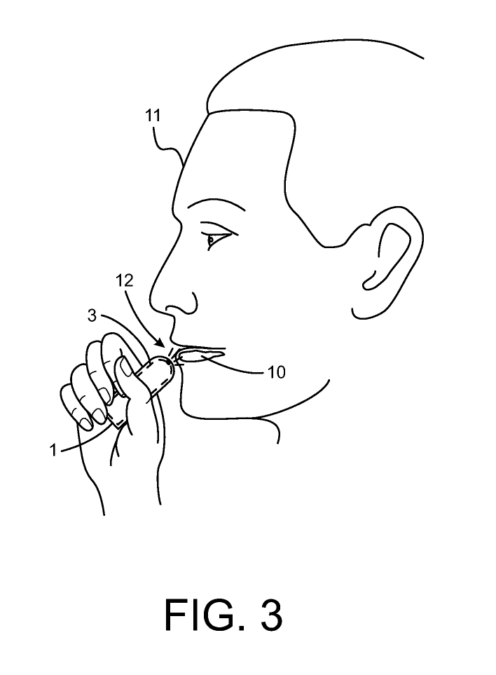

As shown in FIG. 3, an apparatus embodying features of another example of the invention and showing the apparatus with treatment being applied by a person comprises a container 1, treatment composition 3 within the container 1. A person 11 taking the treatment composition 3 expelled from 12 the container 1 and applying it as balm upon the person's lips 10.

The previously described versions of the present invention have many advantages, including that, the method of preventing a body from infection of germs by introducing a microbe killing compound is easily done, the apparatus is small, easy to carry and provides a dual benefit of moisturizing while killing harmful microbes, such as cold or flu germs and bacteria. The particular shapes and configurations of the apparatus as set forth above are preferred because of the above mentioned advantages, but are not required in all versions of the invention.

Although the present invention has been described in considerable detail with the reference to certain preferred versions thereof, other versions are possible. For example, the container might be simply a jar with a covered opening, and provide the ease of use of a tube without the need for applying pressure to the tube to force expellant from the container as the embodiment depicted in FIG. 1. Therefore, the spirit and scope of the appended claims should not be limited to the description of the preferred versions contained herein.

The reader's attention is directed to all papers and documents which are filed concurrently with this specification and which are open to public inspection with this specification. The contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalents or similar features.

What I claim is:

1. An apparatus for applying health treatment to lips, the apparatus comprising:
(a) a container;
(b) the container having an expelling part;
(c) a treatment composition;
(d) the treatment composition being substantially located within the container;
(e) the expelling part having at least one opening;
(f) the treatment composition being sufficiently made of permanently charged ions and having germ killing capabilities;
(g) the treatment composition providing protection from the entry of germs to a living being when applied to the lips of the living being.

2. The apparatus of claim 1, wherein the treatment composition is expellable through the at least one opening.

3. The apparatus of claim 1, wherein the treatment composition is Cetylpyridinium chloride combined with a lip moisturizer.

4. A method of protecting a biological body from unwanted microbes, the method comprising:
(a) obtaining a container that has within it a positively charged composition;
(b) bringing the container into contact or close proximity with at least one outer boundary of a mouth;
(c) expelling the composition onto the at least one outer boundary of a mouth.
